(19) United States
(12) Patent Application Publication
(10) Pub. No.: US 2023/0310442 A1
(43) Pub. Date: Oct. 5, 2023

(54) POLY HYDROXY OLIGOMER COATED DOLUTEGRAVIR AQUASOMES AND METHOD THEREOF

(71) Applicant: Varalakshmi MUMMIDI, Kakinada (IN)

(72) Inventors: Varalakshmi MUMMIDI, Kakinada (IN); Pavani GOVADA, Avanigadda (IN); Mastanamma SK, Guntur (IN)

(21) Appl. No.: 18/023,403

(22) PCT Filed: Aug. 27, 2021
(86) PCT No.: PCT/IN2021/050832

§ 371 Date: Feb. 27, 2023

(30) Foreign Application Priority Data
Aug. 28, 2020 (IN) 202041037143

(51) Int. Cl. A61K 31/5365; A61K 9/50; A61K 47/02; A61K 47/26; A61P 31/22

(52) U.S. Cl. CPC A61K 31/5365; A61K 9/5078; A61K 9/5089; A61K 47/02; A61K 47/26; A61P 31/22

(57) ABSTRACT

The present invention relates to nano particulate carrier system for drug delivery, particularly to Aquasome formulation as a drug delivery system. The invention discloses poly hydroxyl oligomer coated Dolutegravir aquasomes formulation with enhanced solubility. The aquasome formulation comprises of Inorganic core of calcium phosphate Ca₃(PO₄)₂, Carbohydrate or polyhydroxy oligomers comprising Sucrose, Lactose or Trehalose, wherein the inorganic ceramic core is coated by outer sugar or carbohydrate layer and the drug is adsorbed on the sugar or carbohydrate layer to form an aquasome with core: sugar coating: drug is 3:3-6:1 by weight and an average size of 20-70 nm.

### FIELD OF INVENTION

The invention relates to nano particulate carrier system for drug delivery. Particularly the invention relates to Aquasomes as a drug delivery system. The invention discloses poly hydroxy oligomer coated Dolutegravir aquasomes formulation with enhanced solubility. Additionally, the invention relates to the method of preparation of poly hydroxy oligomer coated Dolutegravir aquasomes.

### BACKGROUND OF INVENTION

Aquasomes are nano particulate carrier system but instead of being simple nanoparticle these are three layered self-assembled structures, comprised of a solid phase nanocrystalline core, coated with oligomeric film on which biochemically active molecules are adsorbed with or without modification. Aquasomes are like “bodies of water” and their water like properties protect and preserve fragile biological molecules, and this property of maintaining conformity as well as high degree of surface exposure is exploited in targeting of bioactive molecules like peptide and protein hormones, enzymes, antigens and genes to specific sites. These three layered structures are self-assembled by non-covalent and ionic bonds. These carbohydrate stabilize nanoparticles of ceramic are known as “aquasomes”. The pharmacologically active molecule incorporated by co-polymerization, diffusion or adsorption to carbohydrate surface of preformed nanoparticles. Aquasomes discovery comprises a principle from microbiology, food chemistry, biophysics and many discoveries including solid phase synthesis, supramolecular chemistry, molecular shape change and self-assembly.

The API is the sodium salt of dolutegravir. It is very slightly hygroscopic and contains 2 stereogenic carbon centres. The API is manufactured as a pure enantiomer: sodium (4R,12aS)-9-{[(2,4-difluorophenyl)methyl]carbamoyl}-4-methyl-6,8-dioxo-3,4,6,8,12,12a-hexahydro-2H-pyrido[1′, 2′:4,5]pyrazino[2, 1-b][1,3]oxazin-7-olate. Extensive spectral studies, including 1H, 13 C and 19 F with various techniques, have been provided in support of the structure and absolute configuration of the API. Dolutegravir sodium is critically insoluble (of BCS low solubility across the physiological pH range), hence particle size distribution (PSD) and polymorphism are considered critical parameters and form part of the FPP manufacturer's API specifications. Hence it is critical to improve solubility of Dolutegravir sodium for improved drug delivery.

Accordingly, there is a need to improve the solubility, bioavailability and the thus aid the better targeted delivery of the drug.

### Objects of Invention

It is a primary object of the present invention to provide a nanoparticle drug delivery system for poorly soluble drug.

It is another object of the present invention to provide a three layered ceramic nanoparticle system (aquasomes) for the delivery of drugs.

It is another object of the present invention to provide a nanoparticle for delivery of Dolutegravir drug, specifically ceramic nanoparticles.

It is a particular object of the present invention to provide an Aquasome drug delivery for administration of dolutegravir drug.

It is another object of the present invention to provide a drug delivery system with improved solubility and bioavailability issues by reduction of particle size at room temperature.

It is another object of the present invention to provide Dolutegravir sodium aquasomes by simple and cost effective technique for the treatment of HIV infection.

It is another object of the present invention to provide Dolutegravir sodium aquasomes with increased solubility, dissolution rate and bioavailability.

It is another object of the present invention to provide a method of preparation of ceramic nanoparticles (aquasomes) of dolutegravir with different poly hydroxy oligomers such as sucrose, lactose and trehalose.

It is another object of the present invention to provide a drug delivery system with improved patient compliance and effectiveness of therapy.

### SUMMARY OF THE INVENTION

Thus, according to the present invention, there is provided an aquasome formulation of Dolutegravir, comprising of: Inorganic core of calcium phosphate Ca₃(PO₄)₂, Carbohydrate or polyhydroxy oligomers comprising Sucrose, Lactose or Trehalose, wherein the inorganic ceramic core is coated by outer sugar or carbohydrate layer and the drug is adsorbed on the sugar or carbohydrate layer to form an aquasome, and wherein the ratio of the core: sugar coating: drug is 3:3-6:1 by weight.

It is a primary aspect of the present invention to provide an aquasome formulation of Dolutegravir, comprising of:
inorganic core of calcium phosphate Ca₃(PO₄)₂; and carbohydrate or polyhydroxy oligomers comprising Sucrose, Lactose or Trehalose,
   wherein the inorganic ceramic core is coated by outer sugar or carbohydrate layer and the drug is adsorbed on the sugar or carbohydrate layer to form aquasome, and wherein the ratio of the core: sugar coating: drug is 3:3-6:1 by weight.

It is another aspect of the present invention to provide an aquasome formulation of Dolutegravir, wherein the aquasomes have an average size of 20-70 nm.

It is another aspect of the present invention to provide an aquasome formulation of Dolutegravir, wherein the aquasomes have zetapotential of less than −5 mV to greater than +5 mV when the carbohydrate is Sucrose.

It is another aspect of the present invention to provide an aquasome formulation of Dolutegravir, wherein the aquasomes have zetapotential of less than −20 mV to greater than +20 mV when the carbohydrate is Lactose.

It is another aspect of the present invention to provide an aquasome formulation of Dolutegravir, wherein the aquasomes have zetapotential of less than −30 mV to greater than +30 mV when the carbohydrate is Trehalose.

It is another aspect of the present invention to provide an aquasome formulation of Dolutegravir, wherein the weight of Dolutegravir is in the range of 25-150 mg.

It is another aspect of the present invention to provide a method of preparation of Dolutegravir aquasomes, comprising of steps:
preparation of ceramic core;
sugar coating on the ceramic core; and
adsorption of drug on the coated ceramic,
   wherein the preparation of the ceramic core comprises of reacting equivalent mole ratio (1:1 mole) of disodium hydrogen phosphate with calcium chloride in water, mixing both solutions by sonication of the mixture for 2 hr at RT,
followed by centrifugation to yield the colloidal precipitate, filtration through 0.22 μm; drying at 40° C., 24 h to yield ceramic nanoparticles of Calcium Phosphate represented by the reaction preparation of carbohydrate coat comprises of weighing of sugar and dissolving in water to provide sugar solution;
adding to 150 mg of ceramic nanoparticles taken and 100 ml of sugar solution was added (1:1 or 1:2, core: sugar coat by weight) and sonicated to yield a suspension of nanoparticles in sugar solution;
stirring or mixing using magnetic stirrer for at 25° C. and 800 rpm for 30 min; Centrifuging the resultant solution at 2000 rpm, at 25° C. and 15 min; and
sugar-coated core washed with water and dried at 40° C. in a hot air oven to yield the carbohydrate coated ceramic core, adsorption of drug on the coated ceramic comprises of steps,
   dolutegravir sodium solution of 0.5% w/v in buffer, and addition of the drug solution to weighed quantity of carbohydrate or sugar coated core with stirring at 800-1000 rpm for a time period of 1 hr to 1.5 hrs at a temperature of 25-30° C. resulting in adsorption of drug to the carbohydrate coated nano particles resulting in Dolutegravir.

It is another aspect of the present invention to provide a method of preparation of Dolutegravir aquasomes, wherein dolutegravir sodium solution of 0.5% w/v is prepared in phosphate buffer solution of pH 6.8, adjusted using 1N NaOH.

It is another aspect of the present invention to provide a method of preparation of Dolutegravir aquasomes, wherein centrifugation comprises centrifuging the supernatant at 2000-6000 rpm for a period of 1-1.5 hours.

It is another aspect of the present invention to provide an aquasome formulation of Dolutegravir, wherein the Dolutegravir has an antiviral activity against HSV cells with an IC50 of 18±5 μg/ml.

### BRIEF DESCRIPTION OF DRAWINGS

The annexed drawings show an embodiment of the present invention, wherein:

FIG. 1: Particle size and size distribution of (F4) Sucrose (F5) Lactose (F6) Trehalose coated Dolutegravir Sodium aquasomes.

FIG. 2: DSC thermograms of Pure Dolutegravir Sodium (PD), Sucrose (F4), Lactose (F5) and trehalose (F6) coated Dolutegravir aquasomes.

FIG. 3: FT IR spectra of Pure Dolutegravir Sodium (PD), Sucrose (F4), Lactose (F5) and Trehalose (F6) coated Dolutegravir aquasomes.

FIG. 4: SEM images of Pure Dolutegravir Sodium (PD), Sucrose (F4), Lactose (F5) and Trehalose (F6) coated Dolutegravir aquasomes.

FIG. 5: TEM image of Trehalose (F6) coated Dolutegravir aquasomes.

FIG. 6 Dissolution Profiles of Dolutegravir Sodium Aquasome Formulations.

FIG. 7: Zero Order Plots of Dolutegravir Sodium Aquasome Formulations.

FIG. 8: First Order Plots of Dolutegravir Sodium Aquasome Formulations.

FIG. 9: Hixson Crowell Plots of Dolutegravir Sodium Aquasome Formulations.

FIG. 10a: % Cell viability of Dolutegravir and its aquasomes at HSV cells.

FIG. 10b: % Inhibitory effect of Dolutegravir and its aquasomes at HSV cells

FIG. 11: Antiviral activity of (A) Pure dolutegravir and (B) Trehalose coated Dolutegravir aquasomes by MTT assay.

### DETAILED DESCRIPTION OF THE INVENTION ACCOMPANYING FIGS

Dolutegravir sodium, a BCS Class II drug is an anti-viral agent, which is poorly water soluble with low bioavailability. It needs enhancement of solubility, dissolution rate and to improve its oral bioavailability and therapeutic efficacy. Present invention is aimed at developing three layered ceramic nanoparticles or aquasomes of dolutegravir sodium to explore the relationship between particle size and dissolution rate, and to improve its aqueous solubility and oral bioavailability of the drug.

Drug Profile: Dolutegravir Sodium (Dolutegravir and Tivicay)

IUPAC Name: (3 S,7R)—N-[(2,4-difluorophenyl)methyl]-11-hydroxy-7-methyl-9,12-dioxo-4-oxa-1,8-diazatri cyclo[8.4.0.0³,⁸]tetradeca-10,13-di ene-13-carb oxami de.

Molecular formula: C₂₀H₁₉F₂N₃O₅

Molecular Weight: 419.3788 g/mol

Molecular structure

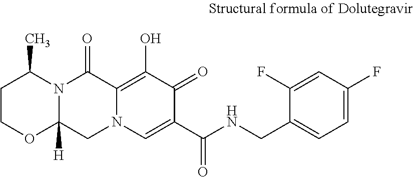 [SMILES: C[C@@H]1CCO[C@H]2Cn3cc(C(=O)NCc4ccc(F)cc4F)c(=O)c(O)c3C(=O)N12]

Physicochemical Properties

Colour: white to off white

Taste & Odour: odorless

Solubility: soluble in water 3.5 mg/mL at 25° C.

Melting point: 190 −193° C.

Particle Size: 5 microns

Mechanism of Action:

Dolutegravir is an HIV-1 antiviral agent. It inhibits HIV integrase by binding to the active site and blocking the strand transfer step to retroviral DNA integration. This is an essential step of the HIV replication cycle and will result in an inhibition of viral activity.

Pharmacokinetics Parameters

Absorption: It exhibits a tₘₐₓ: 0.5-2 hours, Cₘₐₓ, 7.97-14.70 μm, Clearance: 1 L/hrs, Half-life: 14/hrs.

Distribution: Dolutegravir is distributed throughout the body highly protein bound (>98.9%) to human plasma proteins.

Metabolism: Dolutegravir is primarily metabolized by UGT1A1

Elimination: drug eliminated 53% through feces and 32% from urine

Therapeutics Uses: Dolutegravir used in the treatment of HIV infection in used in treatment of other integrase strand inhibitors.

The present work was aimed at developing three layered ceramic nanoparticles or aquasomes of dolutegravir sodium with an objective to reduce the particle size by improve the solubility, dissolution rate, and oral bioavailability of the drug.

Preformulation studies of dolutegravir sodium were performed to know the physical appearance and organoleptic properties. The observation results show that, dolutegravir sodium (DGS) is an amorphous powder and solubility in water was 1.60 μg/mL. Melting point of DGS was observed to be 190-193° C., which complies with reported melting range i.e. 180-190° C.

Aquasome Formulation of Dolutegravir

An embodiment of the present invention provides an aquasome drug delivery system for the drug Dolutegravir. It comprises of three-layered structures comprising of ceramic core, sugar or carbohydrate coating on the core and drug adsorbed layer on the carbohydrate coating. The aquasome formulation comprises of an inorganic core, prepared from disodium hydrogen phosphate with calcium chloride to yield the colloidal precipitate, coated with sugar comprising of Sucrose, Lactose or Trehalose. Different Formulations are prepared wherein the coat.

Materials

Dolutegravir Sodium was gift sample from Eurobond Pharma Pvt. Ltd, India, Disodium hydrogen phosphate from Ozone internationals, Maharashtra, Calcium chloride from Qualigens fine chemicals, India. Sucrose from CDH laboratory, India, Lactose mono hydrate from Finer, Ahmedabad. Trehalose from Kemphasol, Mumbai, All other materials were used by the manufacturers were of Pharmacopeial or analytical grade.

Formulation Design of Dolutegravir Aquasomes:

The three-layered structures are prepared by a three-step procedure, consisting of an inorganic core formation, which will be coated with sugar forming the poly hydroxylase core that will be finally loaded with dolutegravir sodium, a poorly soluble drug.

Dolutegravir aquasomes were prepared by three steps:
1. Preparation of ceramic core
2. Sugar coating on the ceramic core
3. Adsorption of drug on the coated ceramic

Step 1: Preparation of Ceramic Core

The cores were prepared by disodium hydrogen phosphate with calcium chloride to yield the colloidal precipitate with little modification. Based on the reaction stoichiometry, equivalent moles were reacted in a reaction volume of 120 mL specifically, disodium hydrogen phosphate (1 mole=8.90 g) and calcium chloride (1 mole=7.35 g) were taken in 60 mL of water each separately and mixed. A bath sonicator was used for sonication of the mixture for 2 h at room temperature. Following sonication, it was centrifuged at room temperature and 6000 rpm for 1 h. After centrifugation, supernatant was decanted; the precipitate was washed thrice with double-distilled water. The precipitate was resuspended in distilled water (50 mL) and then filtered through a membrane filter pore size 0.22 μ of nitrocellulose. The core was dried at 40° C., 24 h to get ceramic nanoparticles. After drying, the percentage yield was calculated. The chemical reaction involved is as follows,

3Na₂ HPO₄+3CaCl₂→Ca₃(PO₄)₂+6 NaCl+H₃PO₄

Step 2: Sugar Coating on the Ceramic Core Particles

The prepared core particles were coated with polyhydroxy oligomer by adsorption method using sonication. About 150 mg or 300 mg of sugar (Sucrose/Lactose/Trehalose) was weighed and dissolved in 100 ml of double-distilled water as shown in Table 1. In a separate beaker, 150 mg ceramic core was taken and 100 ml of sugar solution was added (1:1 or 1:2, core: sugar coat) and sonicated for 40 min using sonicator. This suspension was shaken or mixing with magnetic stirrer for 30 min at 25° C. and 800 rpm. Here, acetone (non-solvent, 1 mL) was added to the suspension and kept aside for some time. Then, the solution was centrifuged 2000 rpm, at 25° C. and 15 min. The supernatant was decanted off, and the sugar-coated core was washed twice with water and dried at 40° C. for 24 h in a hot air oven sucrose-coated core.

Step 3: Adsorption of Drug on the Sugar-Coated Ceramic Core

Dolutegravir sodium solution of 0.5% w/v (phosphate buffer solution at pH 6.8, and few drops of 1 N NaOH) was added to volumetric flasks containing an accurately weighed amount of sugar-coated core. The flasks were stoppered and shaken vigorously in magnetic stirrer 800 rpm for 1 hr at room temperature. Ceramic nanoparticles (Aquasomes) were filtered through 0.22 μ filter using vacuum pump and dried at 40° C. for 24 h.

The aquasomes or ceramic nanoparticles of Doltegravir, comprises of a ceramic core: sugar: drug in weight proportions 150 mg: 150-300 mg: 50 mg, this is an exemplification of the present invention i.e. the weight ratio is 3:3-6:1 by weight.

TABLE 1
Formulation Design of Dolutegravir Aquasomes

| Ingredients | F1 | F2 | F3 | F4 | F5 | F6 |
|---|---|---|---|---|---|---|
| Dolutegravir Sodium (mg) | 50 | 50 | 50 | 50 | 50 | 50 |
| Sucrose (mg) | 150 | — | — | 300 | — | — |
| Lactose (mg) | — | 150 | — | — | 300 | — |
| Trehalose (mg) | — | — | 150 | — | — | 300 |
| Ceramic core (mg) | 150 | 150 | 150 | 150 | 150 | 150 |

Characterization of Dolutegravir Aquasomes:

1. The Aquasomes were characterized by parameters Entrapment efficiency and Drug loading. Entrapment efficiency is the percentage of actual amount of drug entrapped in the carrier relative to the initial amount of loaded drug. The % entrapment efficiency is calculated by:

% Entrapment efficiency=[(W₁−W₂)/W₁]*100

W₁=total amount of the drug used in preparation

W₂=amount of the drug

For theoretical drug loading it was assumed that entire drug gets entrapped in sugar coated ceramic core. For practical drug loading, an accurately weighed 10 mg of aquasomes were dissolved in 10 mL of pH 6.8 phosphate buffer. Then the solution was transferred to 100 mL of 0.05 N NaOH solution and sonicated for 20 min. Then, the solution was measured the absorbance at 259.8 nm by UV-Vis spectrophotometer.

$$\% Drug loading = \frac{[ wt . of the loaded drug - wt . of unentrapped drug ]}{Total wt of aquasomes} \times 100$$

TABLE 2
Drug Entrapment efficiency and Drug Loading
of Dolutegravir Sodium Aquasomes

| | Drug Entrapment | Drug Loading |
|---|---|---|
| Formulations | Efficiency (%) | (%) |
| F1 | 90.01 ± 0.01 | 4.45 ± 0.05 |
| F2 | 85.22 ± 0.76 | 4.20 ± 0.12 |
| F3 | 92.13 ± 0.06 | 4.54 ± 0.01 |
| F4 | 91.43 ± 0.05 | 4.49 ± 0.09 |
| F5 | 89.32 ± 0.03 | 4.40 ± 0.08 |
| F6 | 93.04 ± 0.56 | 4.59 ± 0.07 |

Note:
All values are expressed as mean ± SE, n = 3

Drug Entrapment Efficiency and % Drug Loading of different aquasome formulations was found to be 92.13±0.06 to 93.04±0.56 and 4.54±0.01 to 4.59±0.07 respectively. The highest entrapment efficiency and % drug loading was found in terhalose coated aquasomes of F6 formulation, which was further evaluated for particle size, zeta potential, morphological studies and in vitro drug release study.

2. Particle Size and Zeta Potential of Dolutegravir Sodium Aquasomes:

The particle size and zeta potential of the dolutegravir aquasomes were determined using Microtrac zetatrac nano technology particle size and charge measurement analyzer (Zetatrac, S/N: W3231, USA). The sample solution was prepared by hydration of aquasomes with water. As shown in instrument parameters Table 3, the sample was taken in disposable sizing cuvettes for particle size and zeta potential analysis. The polydispersity index (PDI) was determined as a measure of homogeneity of the particles. Zetatrac was controlled by microtrac FLEX operating software to generate full characterization data on zeta potential, particle size and size distribution.

TABLE 3
Zetatrac instrument parameters for particle
size and Zetapotential Analysis

| S. No. | Parameters | value | |
|---|---|---|---|
| 1 | Fluid | Water | |
| 2 | Viscosity | 0.869 | cp |
| 3 | Cell Temperature | 26.07° | C. |
| 4 | Dielectric constant | 79 | |
| 5 | Dispersant pH | 7 | |
| 6 | Reflected Pwr (uw) | 1.50 | |
| 7 | Scattering model | Live-Meas | |
| 8 | Loading Index | 0.845 | |
| 9 | Conc. Index | 0.0377 | |
| 10 | Field Strength | 5.0 | kV/m |
| 11 | Conductivity | 143 | uS/cm |

TABLE 4
Particle size and zeta potential of Dolutegravir Sodium Aquasomes

| | | | | Poly | |
|---|---|---|---|---|---|
| | | Particle size | | Dispersity | Zeta |
| | Carbohydrates | (nm) | | Index | potential |
| Formulations | used in Aquasomes | d50 | d90 | (PDI) | (mV) |
| F4 | Sucrose | 44.3 | 35.81 | 0.078 | −11.1 |
| F5 | Lactose | 25.7 | 21.83 | 0.047 | −22.8 |
| F6 | Trehalose | 37.0 | 31.94 | 0.042 | −31.8 |

TABLE 5
Particle size distribution values
of Dolutegravir Sodium Aquasomes

| | |
|---|---|
| Percentile of | Particle size distribution (nm) |

| | Dolutegravir | F4 | F5 | F6 |
|---|---|---|---|---|
| S. No. | Aquasomes (%) | Sucrose | Lactose | Trehalose |
| 1 | 10 | 69.50 | 37.11 | 52.36 |
| 2 | 20 | 58.09 | 32.05 | 45.68 |
| 3 | 30 | 52.09 | 29.16 | 41.79 |
| 4 | 40 | 47.58 | 25.71 | 39.05 |
| 5 | 50 | 44.28 | 27.71 | 37.00 |
| 6 | 60 | 41.86 | 24.67 | 35.50 |
| 7 | 70 | 39.61 | 23.76 | 34.28 |
| 8 | 80 | 38.03 | 22.83 | 33.11 |
| 9 | 90 | 35.81 | 21.83 | 31.94 |
| 10 | 95 | 34.08 | 21.05 | 31.21 |

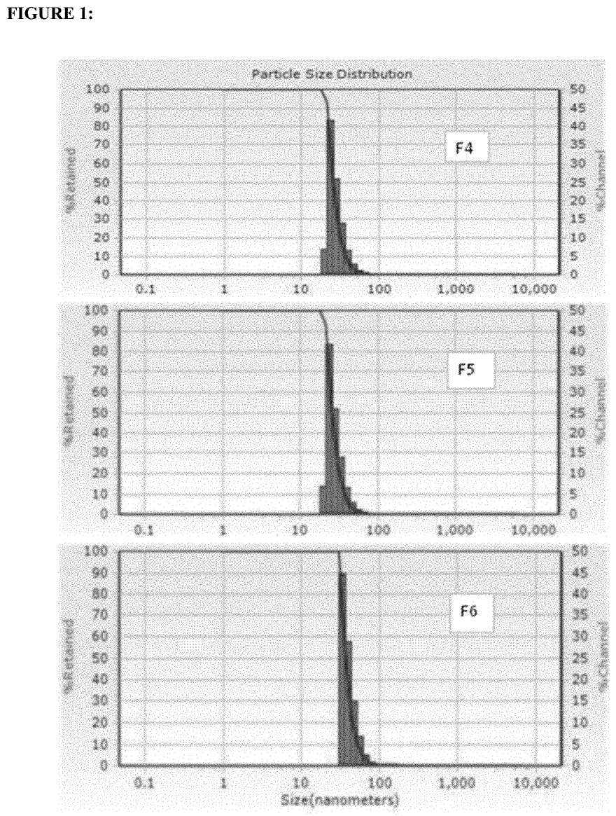

Particle Size and Size Distribution (FIG. 1)

Particle size of dolutegravir aquasomes was determined by Microtrac Zetatrac particle size analyzer. Particle size and size distribution values of the formulations were shown in Table 4 and 5 and FIG. 1. Particle size plays key role in solubility, dissolution rate and bioavailability of the drug. Smaller the particle size greater the dissolution rate. The formulations comprising, F4, F5 and F6 are preferred compositions with three sugars of interest, sucrose, lactose and trehalose. All the three provided particle size of 44.28, 27.71 and 37.00 nm respectively. The most optimized formulation was F6, comprising trehalose coated aquasomal formulation (F6) had a mean (z-average) particle size of 37.0 nm and poly dispersity index (PDI) was found to be 0.042, which indicates the particles are in uniform distribution. An increase or decrease in the particle size of the drug in a formulation can affect its in vitro release and subsequently its bioavailability.

Zeta Potential is an important tool for understanding the surface of the nanoparticle and predicting the stability of the nanoparticles in a solution. It was determined by using Microtrac Zetatrac analyzer. The zeta potential is potential at the hydrodynamic shear plane and can be determined from particle mobility and under electric field. The mobility will depend on surface charge and electrolyte concentration. For molecules and particles that are small enough, a high zeta potential will confer stability i.e., the particles will resist aggregation. When the potential is small, attractive forces may exceed this repulsion and the particles tend to agglomeration. Drug particles dispersed within a liquid continuous medium are stabilized by steric and electrostatic mechanisms, or by a combination of both (i.e., electrostatic mechanism) via carbohydrate. Zeta potential of the dolutegravir aquasomal formulations in Table 4, for Sucrose is −11.1 mV (±5 mV to ±15 mV range), for Lactose is −22.8 mV (±20 mV to ±30 mV range) and for trehalose is −31.8 mV (range ±30 mV to ±40 mV). In general, nanoparticles with zeta potential values greater than +30 mV or less than −30 mV have high degrees of stability. Dispersions with less than +25 mV or greater than −25 mV zeta potential value will eventually agglomerate due to interparticle interactions, including vander Waals and hydrophobic interactions, and hydrogen bonding. The sucrose (F4) and lactose (F5) coated aquasomal formulations are well within the acceptable range of zeta potential for stability, but the optimized trehalose (F6) coated dolutegravir aquasomes was more stable because, greater the zeta potential value greater the stability of the aquasomes.

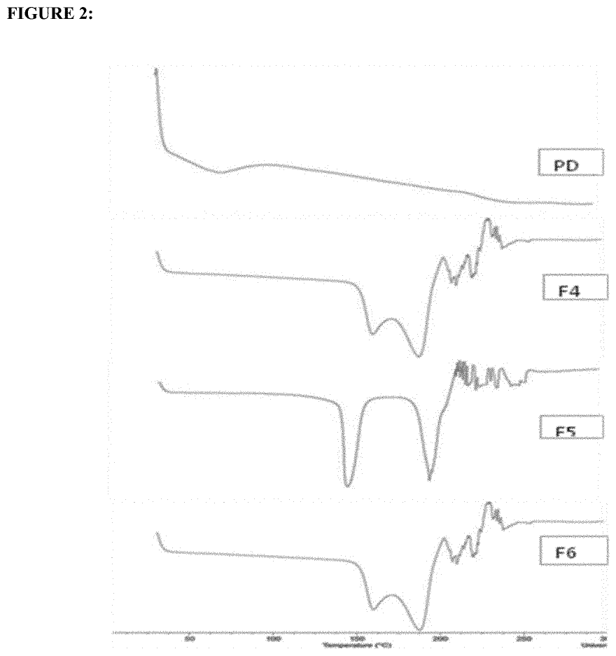

3. Differential Scanning Calorimetry (DSC) Analysis: (FIG. 2)

DSC theromograms of the pure dolutegravir sodium and polyhydroxy oligomers of sucrose, lactose and trehalose coated dolutegravir aquasome formulations were recorded on DSC Q20 model, TA Instrument. Samples about 10 to 15 mg was sealed into aluminium pan and scanned at the heating rate of 10° C./min from 50-300° C. under the nitrogen gas stream. Temperature calibrations were performed using indium as standard. An empty pan sealed in the same way as the sample was used as a reference. The DSC thermograms are shown in FIG. 2.

As illustrated in FIG. 2, The DSC curve of dolutegravir sodium had no sharp endothermic peak at 180.0 to 190.0° C. corresponding to its melting point because of dolutegravir sodium was an amorphous state (PD). Sucrose, lactose and trehalose coated dolutegravir aquasomes were showed (F4, F5 and F6) endothermic peaks were observed at 180.0 to 190.0° C. In the thermograms of the aquasomal formulations, the intensity (or height) of dolutegravir endothermic peak at 190.0 to 192.0° C. was increased than pure dolutegravir and polyhydroxy oligomers like sucrose, lactose and trehalose endothermic peaks were also observed. Hence there was no interaction of dolutegravir sodium with polyhydroxy oligomers.

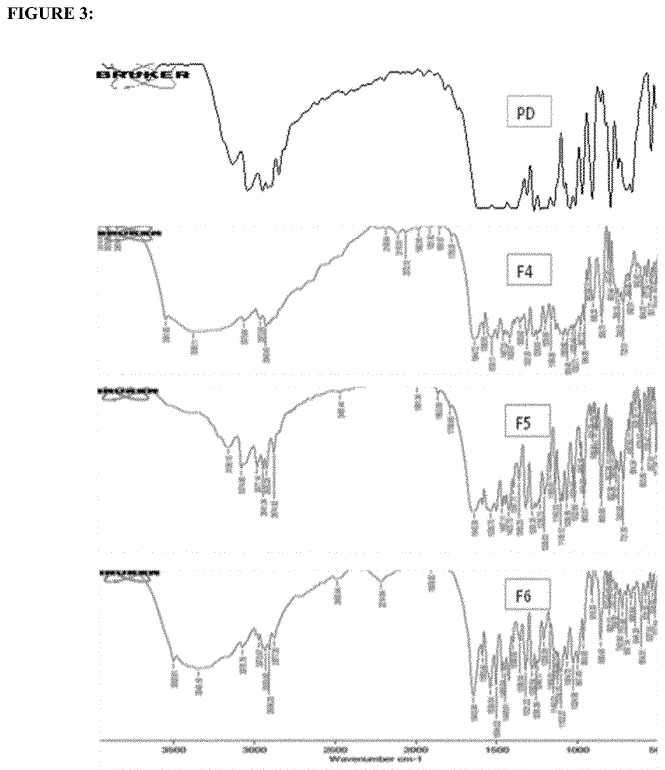

4. Fourier—Transform Infrared Spectroscopy: (FIG. 3)

Fourier transforms infrared spectral spectroscopy of pure dolutegravir sodium and various polyhydroxy oligomers (sucrose, lactose and trehalose) of dolutegravir aquasome formulations were mixed with IR grade potassium bromide in the ratio of 1:100 and pellets were prepared by applying 10 metric ton of pressure in hydraulic press. The pellets were then scanned over range of 4000-400 cm⁻¹ in FTIR spectrometer (BRUKER-Germany) and the results are shown in FIG. 3.

The main absorption bands of drug were observed as O—H stretching at 3155, C═C bending at 1503, —C—H bonding at 1211 and ═CH₂ rocking at 884 were present in spectra that indicating compatibility. It shows that there was no significant change in the chemical integrity of the drug.

In Drug-excipients compatibility studies the peaks observed in FT-IR of mixture of dolutegravir and aquasome formulations at 3349.93 cm⁻¹, and 1635.55 cm⁻¹. There was no major shifting in the frequencies of above said functional groups of which indicates that there was no chemical interaction between dolutegravir and excipients which were used in the formulation.

TABLE 6
Interpretation of FTIR of Pure dolutegravir
and Aquasomal formulations

| Wavenumber(cm⁻¹) | | | | |
|---|---|---|---|---|
| Pure | Aquasomal formulations | | | |
| Dolutegravir | Sucrose | Lactose | Trehalose | Functional |
| Sodium (PD) | (F4) | (F5) | (F6) | Group |
| 3155 | 3069 | 3073 | 3180 | Usually sharp O—H |
| 2975 | 2973 | 2925 | 2842 | CH₃, CH₂&CH |
| 2941 | 2803 | 2930 | 2750 | CH, Stretch |
| 2874 | 1541 | 2974 | 2945 | C═C |
| 1503 | 1456 | 1530 | 1576 | ═CH₃ |
| 1425 | 1274 | 1470 | 1454 | C—H |
| 1211 | 1089 | 1290 | 1274 | —C—H, Bending |
| 1028 | 849 | 1924 | 1052 | —C—N |
| 884 | 605 | 890 | 872 | ═CH₂ |

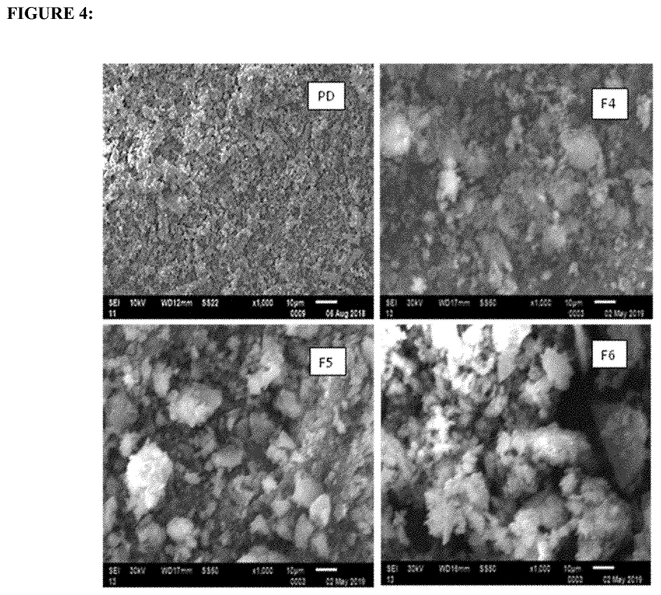

5. Scanning Electron Microscopy (SEM): (FIG. 4)

Scanning electron microscopy was used to study the surface characteristics of pure dolutegravir sodium and various polyhydroxy oligomers (sucrose, lactose and trehalose) of dolutegravir aquasomes were observed using scanning electron microscope, Philips XL-30 SEM (Basel, The Netherlands). Samples were placed on a carbon specimen holder, and then coated with a thin gold layer using a sputter coater unit. The scanning electron microscope was operated at 30 kV acceleration voltage and the images are shown in FIG. 4.

SEM was used to study the microscopic characters of dolutegravir sodium and their carbohydrate coated aquasomal formulations. The SEM photographs of pure dolutegravir and aquasomal formulations (F4, F5 and F6) are shown in FIG. 4. SEM of dolutegravir sodium powder showed amorphous of different sizes with smooth surfaces. In aquasomal formulations, the smaller particles were seen to have adhered to the surfaces of larger ones. In the SEM of trehalose coated dolutegravir aquasomes, the particles are having smooth surfaces with different sizes were noticed. These microscopic observations indicated a good physical interface of drug particles with different polyhydroxy oligomers. Although SEM technique is inadequate to conclude aquasomes formation, the SEM micrographs support the formation of polyhydroxy oligomers entrapping the drug particles.

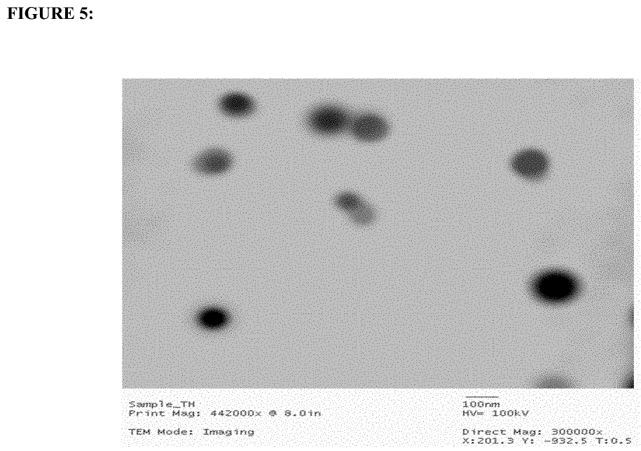

Transmission Electron Microscopy (TEM) studies: (FIG. 5)

Transmission electron microscopy (TEM) was used to evaluate the shape of the aquasomes and adsorption of drug on the sugar-coated ceramic core. A Philips CM 10 transmission electron microscope was operated at 100 kV acceleration voltage and particle size was measured using NIH image software. The trehalose coated aquasomes, at a concentration of 0.5% (w/v) of aquasome, were sprayed on Formvar-coated copper grids and air-dried before observation and the image shows in FIG. 5.

TEM studies were very useful in determining shape and morphology of aquasomal formulations. It determines the particle size with or without staining. TEM uses electron transmitted through the specimen and has much higher resolution than SEM. TEM photomicrograph of the optimized trehalose coated aquasomes (F6) were spherical in shape are reported in FIG. 5 and confirm their previously ascertained sizes (<100 nm) with a rather uniform distribution and adsorption of drug on the sugar-coated ceramic core.

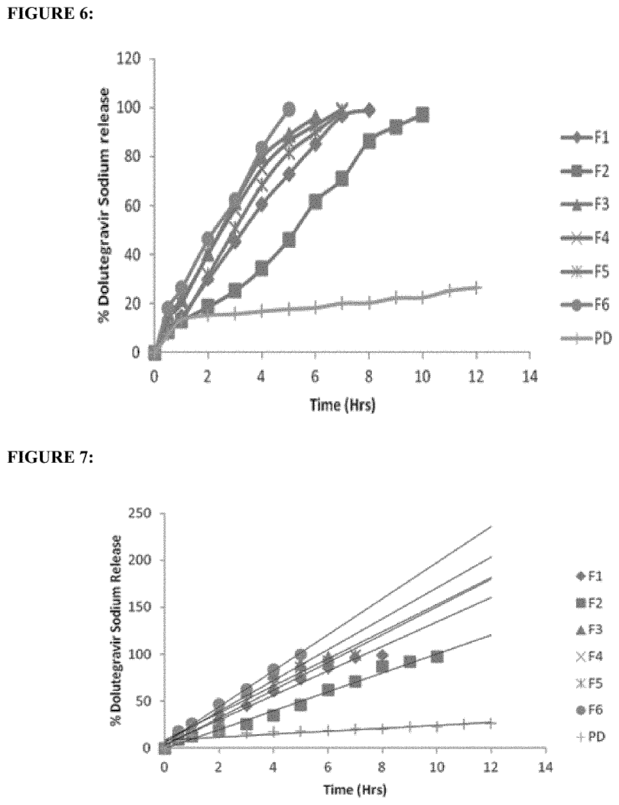

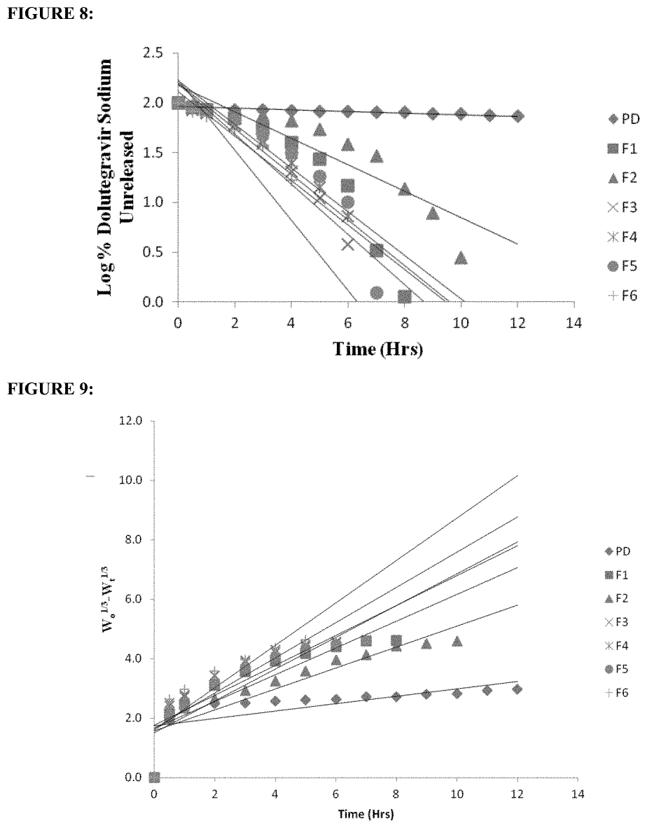

In vitro drug dissolution studies 01 In vitro dissolution studies of the pure dolutegravir (PD) and its aquasomal formulations (F1 to F6) were carried out using USP-Type II dissolution apparatus. 900 ml of pH 6.8 phosphate buffer was used as dissolution media and temperature was maintained at 37° C.±0.5° C. with paddle rotation speed at 50 rpm. Aliquots of 5 ml were withdrawn at various intervals and were replaced with same quantity of fresh dissolution medium to maintain the sink condition. Samples were filtered through wattman filter paper and analysed UV—Vis spectrophotometrically at 259.80 nm. The dissolution experiments were conducted in triplicate and the cumulative percentage of drug release was calculated. Percentage of drug release was showed in the Table 7 and FIG. 6. Dissolution efficiency (DE) values were calculated as per Khan¹ and T50 (time taken for 50% dissolution) values were recorded from the dissolution profiles. The dissolution parameters are summarized in Table 8.

6. In Vitro dissolution of Dolutegravir Sodium Aquasomes

A comparative in vitro drug release study was performed in pH 6.8 phosphate buffer for pure dolutegravir sodium (PD) and all designed formulations (F1-F6), the data was shown in Table 7. The dissolution experiments were conducted in triplicate. Dissolution efficiency (DE₅) values were calculated as per Khan¹. T₅₀ (time taken for 50% dissolution) values were recorded from the dissolution profiles. The dissolution parameters are summarized in Table 8.

The K₀ and DE₅ values of aquasomal formulations exhibited higher rates of dissolution than PD may be due to reduction of particle size of the dolutegravir sodium in aquasomes. Increase in the surface area and dissolution rate may be attributed to, the reduced particle size of drug at the time coated with soluble material like polyhydroxy oligomers (carbohydrates) which is earlier discussed under the Table 4 with average size 20-50 nm

All aquasomal formulations exhibited higher rates of dissolution and DE values than pure dolutegravir, indicating rapid and higher dissolution of carbohydrate coated dolutegravir aquasomes. The K₁ and DE₅ values increased as the proportion of polyhydoxy oligomers was increased in each case. The increase in K₁ (no. of folds) with various aquasomes is shown in Table 8. Trehalose coated dolutegravir aquasomes (F6) gave higher enhancement in the dissolution rate and efficiency when compared to sucrose (F4) and lactose (F5) coated aquasomes. The higher dissolution rates and DE values observed with trehalose coated aquasomes may be due to the better drug-carbohydrate coating during the aquasomal formulation process.

The dissolution data of formulations PD and dolutegravir aquasomes were fitted into mathematical models such as zero order, first order and Hixson Crowell models kinetics and the plots were shown in Table No: 8. The release kinetics of pure drug (PD) and dolutegravir aquasomes follows zero order as well as Hixson Crowell model because the values of regression coefficient obtained for zero order release profiles are higher as compared to first order kinetics. Hixson Crowell kinetic plot of F6 (r=0.722) shows higher correlation coefficient value than PD (r=0.443). The cube root dissolution rate constant (K_{H}) of Hixson Crowell values of PD is 0.124 and optimized formulation F6 is 0.713. During dissolution, the radius of the particle, mass of the particle was changed. Hence, the drug release by dissolution is high with the change in surface area and diameter of the particles as illustrated in Table No: 7

Fit Factor Analysis (f1 and f2)

Applying fit factor tests (f₁ and f₂), under appropriate test conditions a dissolution profile may be used to characterize a product more precisely than a single point dissolution test. Dissimilarity factor (f1) and similarity factor (f2) were calculated. The values (f1=289.7 and f2=15.42) shows that there is no similarity between both the profiles. Therefore, it may be concluded that optimized aquasome formulation (F6) not only has superior dissolution profile than pure drug and but also has much better release profile when compared to pure dolutegravir (PD).

Statistical analysis by unpaired t-test was performed to test whether the difference in mean dissolution efficiency values at 5 h in pH 6.8 phosphate buffer were observed between pure dolutegravir (PD) and optimized formulation (F6, trehalose coated dolutegravir aquasomes) was significant or not. The analysis revealed that the difference between the methods was significant at P<0.05. The absolute value of the calculated ‘t’(117.74)>table ‘t’ (0.0001), this difference is considered to be extremely statistically significant between pure dolutegravir (PD) and optimized formulation (F6).

Two way analysis of variance was conducted to test whether the difference in mean dissolution efficiency values at 5 h observed between the three polyhydroxy oligogmers (sucrose, lactose and trehalose) and its ratio (core: oligomer coat, 1:1 and 1:2) of aquasomal formulations were significant or not, The analysis revealed that the difference between the three types of polyhydroxy oligogmers (F=2.72) and its ratios (F=7.12) of aquasomal formulations were also statistically significant at p<0.05. There was interaction effect between the types of polyhydroxy oligomers and its ratio influenced on dissolution rate. Hence, the ratio of 1:2:0.3, trehalose coated dolutegravir aquasomes was the best among the polyhydroxy oligomers coated aquasomes.

TABLE 7
In Vitro Dissolution of Dolutegravir Sodium Aquasome formulations

| Time | % of Dolutegravir Sodium Release | | | | | | |
|---|---|---|---|---|---|---|---|
| (hr) | PD | F1 | F2 | F3 | F4 | F5 | F6 |
| 0 | 0 | 0 | 0 | 0 | 0 | 0 | 0 |
| 0.5 | 7.38 ± 0.12 | 10.01 ± 0.12 | 8.61 ± 0.09 | 15.72 ± 0.11 | 14.24 ± 0.09 | 9.27 ± 0.10 | 18.01 ± 0.08 |
| 1 | 13.45 ± 0.14 | 14.67 ± 0.11 | 12.93 ± 0.02 | 21.65 ± 0.12 | 20.07 ± 0.10 | 13.32 ± 0.12 | 26.22 ± 0.15 |
| 2 | 15.27 ± 0.12 | 29.92 ± 0.09 | 18.75 ± 0.14 | 40.32 ± 0.12 | 40.51 ± 0.16 | 31.72 ± 0.13 | 46.43 ± 0.12 |
| 3 | 15.79 ± 0.19 | 45.21 ± 0.14 | 25.12 ± 0.12 | 61.21 ± 0.12 | 58.45 ± 0.12 | 50.62 ± 0.18 | 62.35 ± 0.13 |
| 4 | 16.92 ± 0.12 | 60.52 ± 0.10 | 34.21 ± 0.16 | 79.85 ± 0.15 | 74.97 ± 0.14 | 68.14 ± 0.19 | 83.45 ± 0.14 |
| 5 | 17.82 ± 0.09 | 72.92 ± 0.11 | 46.12 ± 0.08 | 88.91 ± 0.19 | 85.95 ± 0.18 | 81.67 ± 0.02 | 99.32 ± 0.11 |
| 6 | 18.33 ± 0.10 | 85.27 ± 0.15 | 61.75 ± 0.18 | 96.21 ± 0.12 | 92.72 ± 0.15 | 89.97 ± 0.11 | — |
| 7 | 20.13 ± 0.12 | 96.71 ± 0.11 | 70.87 ± 0.12 | — | 99.18 ± 0.25 | 98.77 ± 0.14 | |
| 8 | 20.27 ± 0.43 | 98.87 ± 0.10 | 86.41 ± 0.13 | | | — | |
| 9 | 22.27 ± 0.12 | — | 92.25 ± 0.11 | | | | |
| 10 | 22.45 ± 0.14 | | 97.12 ± 0.18 | | | | |
| 11 | 25.23 ± 0.13 | | — | | | | |
| 12 | 26.45 ± 0.22 | | | | | | |

Note:
All values are expressed as mean ± SE, n = 3

TABLE 8
Dissolution parameters of Zero, First and Hixson Crowell
kinetics values of Dolutegravir sodium release

| | | | | Kinetics of Drug release | | | | | |
|---|---|---|---|---|---|---|---|---|---|
| | Correlation Coefficient | | | Zero | First | Hixson | | Increase | |
| | Values (r) | | | order | order | Crowell | | in K₁ | |
| | Zero | First | Hixson | (Kₒ) | (K₁) | (K_{H}) | T₅₀ | (folds) | DE₅ |
| Formulation | order | order | Crowell | mg/hr | 1/hr | mg^{1/3}/hr | (hr) | * | (%) |
| PD | 0.812 | 0.845 | 0.443 | 1.566 | 0.018 | 0.124 | 38.5 | — | 14.80 |
| F1 | 0.987 | 0.848 | 0.772 | 13.02 | 0.306 | 0.453 | 2.30 | 17.00 | 37.11 |
| F2 | 0.988 | 0.862 | 0.820 | 10.06 | 0.502 | 0.352 | 1.38 | 27.89 | 23.05 |
| F3 | 0.977 | 0.943 | 0.726 | 16.41 | 0.511 | 0.592 | 1.35 | 28.39 | 37.30 |
| F4 | 0.967 | 0.866 | 0.716 | 14.49 | 0.578 | 0.503 | 1.19 | 32.11 | 40.32 |
| F5 | 0.985 | 0.848 | 0.787 | 14.82 | 0.506 | 0.534 | 1.36 | 28.11 | 41.15 |
| F6 | 0.993 | 0.789 | 0.722 | 19.2 | 0.810 | 0.713 | 1.01 | 45.00 | 54.07 |

*Ratio of K₁ of Aquasomes to K₁ of dolutegravir.

Stability studies were conducted to optimized final formulation F6. The drug content and percentage of drug release from the formulation was satisfactory and any noticeable changes were not observed.

Overall result of these studies reveals that, carbohydrate coated dolutegravir sodium shows good dissolution profile compared to pure drug. The sucrose, lactose, and trehalose coated aquasomes shows fast dissolution, particularly trehalose when compared to other carbohydrates like sucrose and lactose. Thus, aquasomes as potential carriers for the delivery of model hydrophobic drugs.

Antiviral activity was determined by MTT assay

In vitro MTT antiviral assay

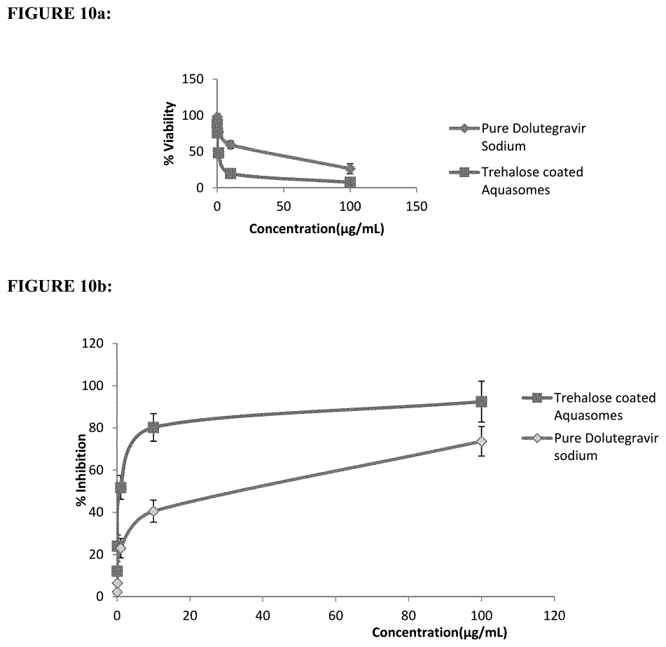

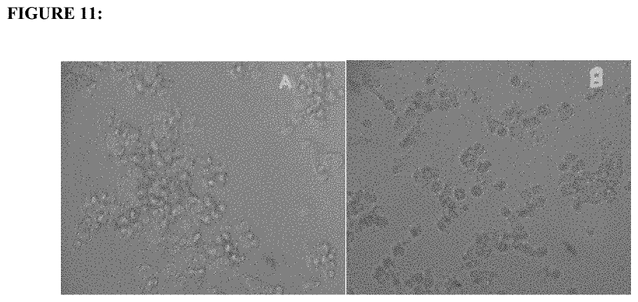

Cells (1×10⁵ cells/ml) were seeded on 96-well tissue culture plates. After a 24 h period of incubation, the medium was removed and replenished with 100 ml of medium containing increasing concentrations of the compounds (serially diluted two fold). As cell control, 100 μl of medium only is added. After three to five days of incubation, the medium was removed and 50 ml of MTT solution (2 mg/ml) was added to each well for 4 h at 37° C. Then, 100 μl of iso-propanol was added to each well in order to dissolve the formazan crystals. After shaking gently the plates for 10 min to dissolve the crystals, the colour reaction was measured in an automated microplate reader at 562 nm. The untreated control was arbitrarily set as 100%. For each compound, the percentage of cell protection/virus inhibition can be calculated as [(Mean OD of control group—Mean OD of treated group)/Mean OD of control group]×100 Dolutegravir pure drug (PD) and it aquasomal formulations (F6) in concentrations 0.01 to 100 μ/mL exhibit antiviral activity against herpes simplex virus (HSV) strains shown in FIGS. 10 and 11. The cell viability after exposing HSV infected vero cells, Dolutegravir produced around 50% inhibition of cell viability with an IC50 value of 57.92±4.6 μg/mL and 18.47±5.4 μg/mL for PD and F6 formulations respectively. Thus, trehalose coated dolutegravir aquasomes (F6) showed 3.13-fold more antiviral activity in comparison to pure dolutegravir sodium.

I claim:

1. An aquasome formulation of Dolutegravir, comprising of:
Inorganic core of calcium phosphate Ca₃(PO₄)₂; and
Carbohydrate or polyhydroxy oligomers comprising Sucrose, Lactose or Trehalose,
wherein the inorganic ceramic core is coated by outer sugar or carbohydrate layer and the drug is adsorbed on the sugar or carbohydrate layer to form aquasome, and wherein the ratio of the core: sugar coating: drug is 3:3-6:1 by weight.

2. The aquasome formulation of Dolutegravir as claimed in claim 1, wherein the aquasomes have an average size of 20-70 nm.

3. The aquasome formulation of Dolutegravir as claimed in claim 1, wherein the aquasomes have zetapotential of less than −5 mV to greater than +5 mV when the carbohydrate is Sucrose.

4. The aquasome formulation of Dolutegravir as claimed in claim 1, wherein the aquasomes have zetapotential of less than −20 mV to greater than +20 mV when the carbohydrate is Lactose.

5. The aquasome formulation of Dolutegravir as claimed in claim 1, wherein the aquasomes have zetapotential of less than −30 mV to greater than +30 mV when the carbohydrate is Trehalose.

6. The aquasome formulation of Dolutegravir as claimed in claim 1, wherein the weight of Dolutegravir is in the range of 25-150 mg.

7. A method of preparation of Dolutegravir aquasomes, comprising of steps:
preparation of ceramic core;
sugar coating on the ceramic core; and
adsorption of drug on the coated ceramic,
wherein the preparation of the ceramic core comprises of reacting equivalent mole ratio (1:1 mole) of disodium hydrogen phosphate with calcium chloride in water, mixing both solutions by sonication of the mixture for 2 hr at RT,
followed by centrifugation to yield the colloidal precipitate, filtration through 0.22 μm;
drying at 40° C., 24 h to yield ceramic nanoparticles of Calcium Phosphate represented by the reaction preparation of carbohydrate coat comprises of weighing of sugar and dissolving in water to provide sugar solution;
adding to 150 mg of ceramic nanoparticles taken and 100 ml of sugar solution was added (1:1 or 1:2, core: sugar coat by weight) and sonicated to yield a suspension of nanoparticles in sugar solution;
stirring or mixing using magnetic stirrer for at 25° C. and 800 rpm for 30 min;
centrifuging the resultant solution at 2000 rpm, at 25° C. and 15 min;
sugar-coated core washed with water and dried at 40° C. in a hot air oven to yield the carbohydrate coated ceramic core; and
adsorption of drug on the coated ceramic comprises of steps,
preparation of Dolutegravir sodium solution of 0.5% w/v in buffer, and addition of the drug solution to weighed quantity of carbohydrate or sugar coated core with stirring at 800-1000 rpm for a time period of 1 hr to 1.5 hrs at a temperature of 25-30° C. resulting in adsorption of drug to the carbohydrate coated nano particles resulting in Dolutegravir.

8. The method of preparation of Dolutegravir aquasomes as claimed in claim 7, wherein dolutegravir sodium solution of 0.5% w/v is prepared in phosphate buffer solution of pH 6.8, adjusted using 1N NaOH.

9. The method of preparation of Dolutegravir aquasomes as claimed in claim 7, wherein centrifugation comprises centrifuging the supernatant at 2000-6000 rpm for a period of 1-1.5 hours.

10. The aquasome formulation of Dolutegravir as claimed in claim 1, wherein the Dolutegravir has an antiviral activity against HSV cells with an IC50 of 18±5 μg/ml.
